(19) United States
(12) Patent Application Publication
(10) Pub. No.: US 2026/0215425 A1
(43) Pub. Date: Jul. 30, 2026

(54) NITRIFICATION INHIBITORS

(71) Applicant: The University of Melbourne, Melbourne, Victoria (AU)

(72) Inventors: Sibel Cansu Yildirim, Melbourne (AU); Uta Wille, Melbourne (AU)

(21) Appl. No.: 19/107,386

(22) PCT Filed: Aug. 28, 2023
(86) PCT No.: PCT/AU2023/050830

§ 371 Date: Feb. 27, 2025

(30) Foreign Application Priority Data
Sep. 2, 2022 (AU) 2022902533

(51) Int. Cl. A01N 43/647; A01P 1/00; C05C 3/00; C05C 9/00; C05G 3/90; C07D 249/04

(52) U.S. Cl. CPC A01N 43/647; A01P 1/00; C05C 3/00; C05C 9/00; C05G 3/90; C07D 249/04

(57) ABSTRACT

A compound of Formula (I) when used for inhibiting nitrification: wherein: R¹ is CH₂CΞCH; R² is methyl; and R³ is H.

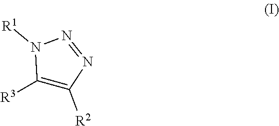

### RELATED APPLICATIONS

This application is a U.S. National Stage filing under 35 U.S.C. 371 of International Patent Application No. PCT/AU2023/050830, filed Aug. 28, 2023, and titled NITRIFICATION INHIBITORS, which claims priority to Australian Patent Application No. 2022902533, filed Sep. 2, 2022, each of which is incorporated herein by reference in its entirety.

### FIELD

The invention relates to compounds which inhibit nitrification and to methods of inhibiting nitrification.

### BACKGROUND

This application claims priority from Australian Provisional Application No. 2022902533, filed on 2 Sep. 2022 and entitled “Nitrification inhibitors”. The entire contents of that application are incorporated herein by reference.

Nitrification is a major cause of nitrogen loss in agriculture. Whereas nitrogen is a nutrient for plants, it is equally a nutrient for soil microorganisms. When nitrogen fertilisers are applied to soil, only a small portion ends up in plants. The vast majority is taken up by microorganisms in the soil, which convert nitrogen into higher oxidised species, such as nitrite (NO₂⁻), nitric oxide, and nitrate (NO₃⁻) that are not readily available to plants. Additionally, nitrous oxide (N₂O) is produced through nitrification-denitrification processes. N₂O is a greenhouse gas, having a global warming potential around 300 times greater than carbon dioxide (CO₂). Agriculture currently generates around 90% of the total amount of N₂O globally.

Due to the chemical transformations of nitrogen fertilisers by soil microorganisms, which compete with plant uptake, farmers are forced to introduce increasingly more of these fertilisers in order to increase crop yield. This practice perpetuates the vicious cycle of nitrogen fertilisation and nitrogen loss to the environment.

Some nitrification inhibitors (NIs) are already commercially available. It is believed that these NIs bind to the key (initial) enzyme ammonia monooxygenase (AMO), which is preserved in bacteria and archaea, and which catalyses the first step of the nitrification. This membrane-bound copper-containing metal oxidase has an as yet unknown structure, as attempts to crystallize this enzyme have so far been unsuccessful. Existing NIs, such as the commercial compounds 3,4-dimethylpyrazole phosphate (DMPP), dicyandiamide (DCD) and the recently discovered 1,4-substituted 1,2,3-triazoles, bind reversibly to AMO. Accordingly, the activity of these NIs is only temporary.

There is therefore a need for a means to prevent the nitrification activity of soil microorganisms that is longer lasting than existing solutions.

### SUMMARY OF THE INVENTION

In a first aspect of the invention there is provided a compound of Formula (I) when used for inhibiting nitrification:

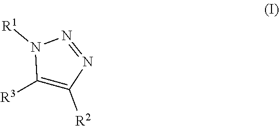

wherein:
R¹ is CH₂C≡CA, wherein A is H, methyl or ethyl;
R² is CₙH₂ₙ₊₁;
R³ is CₘH₂ₘ₊₁;
   and wherein n and m+n are each independently 1, 2, 3, 4, 5 or 6 and m is 0, 1, 2, 3, 4 or 5.

The following options may be used in conjunction with the first aspect, either individually or in any suitable combination.

A may be CₓH₂ₓ₊₁, where x is 0, 1 or 2. It may be H, methyl or ethyl. It may be H. n may be 1. m may be 0.

The compound may bind irreversibly to a nitrification enzyme. It may irreversibly deactivate a nitrification microorganism.

The compound may be such that nitrification activity of Nitrosomonas europaea (N. europaea) or Nitrosospira multiformis (N. multiformis) increases from step (ii) to step (iv) by less than 10% on the following test:
i. ammonium sulphate and the compound are added to cells of a nitrification microorganism, e.g. N. europaea or N. multiformis cells, in sodium phosphate buffer (NaPB);
ii. NO₂ production is measured via a Griess assay;
iii. the cells are then washed with buffer and ammonium sulphate added;
iv. NO₂⁻ production is again measured using a Griess assay.

In one embodiment there is provided a compound of Formula (I) when used for inhibiting nitrification:

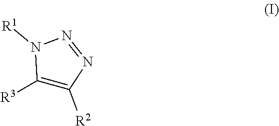

wherein:
R¹ is CH₂C≡CH;
R² is CH₃;
R³ is H;

In a second aspect of the invention there is provided a composition when used for inhibiting nitrification in soil, comprising a compound of Formula (I) as defined in the first aspect and at least one agriculturally acceptable adjuvant or diluent. The composition may further comprise a urease inhibitor.

In a third aspect of the invention there is provided a fertiliser composition comprising a fertiliser and a compound of Formula (I) as defined in the first aspect. The fertiliser may be a urea-based fertiliser or may be an ammonium-based fertiliser. It may further comprise a urease inhibitor.

In a fourth aspect of the invention there is provided a method for reducing nitrification in soil comprising treating the soil with a compound of Formula (I) as defined in the first aspect or a composition as defined in the second aspect.

The following options may be used in conjunction with the fourth aspect, either individually or in any suitable combination.

The soil may be an acidic soil. It may be a mildly acidic soil. It may be a non-acidic soil. It may be mildly alkaline. It may be alkaline. It may be neutral. The treating may be repeated no more than once in any 2-week period.

In a fifth aspect of the invention there is provided a method for inhibiting nitrification activity of a nitrification microorganism, e.g. N. europaea or N. multiformis, comprising exposing said microorganism to a compound of Formula (I) as defined in the first aspect or a composition as defined in the second aspect. The nitrification microorganism may be nitrifying bacteria.

In a sixth aspect of the invention there is provided a method for deactivating a nitrification enzyme comprising exposing said enzyme to a compound of Formula (I) as defined in the first aspect or a composition as defined in the second aspect. The nitrification enzyme may be ammonia monooxygenase (AMO) or may be hydroxylamine oxireductase (HAO). In one embodiment the enzyme is AMO.

In a seventh aspect of the invention there is provided use of a compound of Formula (I) as defined in the first aspect for reducing nitrification in soil, or for inhibiting nitrification activity of a nitrification microorganism (such as nitrifying bacteria, e.g. N. europaea or N. multiformis), or for deactivating a nitrification enzyme.

In an eighth aspect of the invention there is provided use of a compound of Formula (I) as defined in the first aspect in manufacture of a composition for reducing nitrification in soil, or for inhibiting nitrification activity of a nitrification microorganism, or for deactivating a nitrification enzyme.

### BRIEF DESCRIPTION OF DRAWINGS

Certain embodiments of the present invention will now be described, by way of example only, with reference to the accompanying figures, wherein:

FIG. 1 shows the effect of various functional groups on the nitrification inhibition, determined using N. europaea (NE) and N. multiformis (NM). NO₂ production in ammonia oxidizing bacteria (AOB) was determined following the ‘standard nitrification inhibition assay protocol’ described herein. The incubations in NaPB with 1 v/v % DMSO were performed at pH=7.5 and 30° C. with [NH₄⁺]=3 mM, [inhibitor]=0.3 mM (10 mol % of [NH₄⁺]) at 100 rpm for 60 min in the dark. Standard errors were determined from three biological and three technical replicates. No inhibition is indicated as NI.

FIG. 2 shows results of an enzymatic activity recovery assay performed with N. europaea. I) 30 min+Inh indicates the percent activity of AOB treated 30 mins with inhibitor (1.5 mM, 50 mol % of NH₄⁺) in comparison to uninhibited cells. II) ‘30 min−Inh’ shows the percent activity of AOB after the washing steps and re-incubation with (NH₄⁺)₂SO₄. The incubations were performed in NaPB with 1 v/v % DMSO at pH=7.5 and 30° C. with [NH₄⁺]=3 mM, [inhibitor]=1.5 mM (50 mol % of [NH₄⁺]) at 100 rpm for 60 min in the dark. Standard errors were determined from three biological and three technical replicates.

FIG. 3 shows results of an oxygen (O₂) consumption experiment performed with N. europaea. The trace describing the O₂ concentration after addition of (NH₄)₂SO₄ against the time (initial 300-600 seconds) was used as the baseline for the uninhibited cells, whereas the trace describing the consumption in the presence of the inhibitor (605−1800 seconds timespan) was used to determine the rate with inhibitor. The dashed line indicates the predicted [O₂] without inhibitor. All experiments were conducted in triplicate at 20° C. and under constant stirring.

FIG. 4 shows a) ‘mode of inhibition’ and b) ‘dose-response curve’ performed with N. europaea. The incubations were performed in NaPB with 1v/v % DMSO at pH=7.5 and 30° C. with different concentrations of NH₄⁺ and inhibitor at 100 rpm for 60 min in the dark. Standard errors were determined from three biological and three technical replicates.

FIG. 5 shows soil incubation studies performed in soil A with (1) fertiliser ((NH₄)₂SO₄,) 100 mg kg⁻¹, (2) fertiliser+0.5 mol % 4-MPT (4-methyl-1-(prop-2-yn-1yl)-1H-1,2,3-triazole), or (3) fertiliser+2.5 mol % 4-MPT (4) fertiliser+5 mol % 4-MPT (5) fertiliser+5 mol % compound 1 of the applied fertiliser-N, respectively. Standard errors were determined from three biological replicates.

FIG. 6 shows a graph of NO₂ production against NH₄⁺ concentration to determine the mode of inhibition using Michaelis-Menten kinetics.

FIG. 7 shows N₂O production rates over 21 days in soils B-E. (NH₄)₂SO₄ was applied as 50 mg kg⁻¹ soil ‘fertiliser only’. [DMP] and [4-MPT]were 0.5 mol %, 2.5 mol % and 5 mol % of the applied fertiliser-N, respectively. Soil E: Note the different axis scale to include higher N₂O production rates; no N₂O production was detected beyond day 5 for DMP (3,4-dimethylpyrazole) at 5 mol % and all 4-MPT treatments. Data were calculated from three biological replicates.

### DEFINITIONS

As used herein, the term “comprising” means “including”. Variations of the word “comprising”, such as “comprise” and “comprises”, have correspondingly varied meanings. As used herein, the terms “including” and “comprising” are non-exclusive and allow for the presence of other integers, optionally unspecified. As used herein, the terms “including” and “comprising” do not imply that the specified integer(s) represent a major part of the whole. The term “consists essentially of” means that the specified integers are the only integers intentionally present, although other integers, generally minor, may incidentally be present. The term “consists of” means that the specified integers are the only integers present.

The meaning of general words is not limited by specific examples introduced by “include”, “includes”, “including”, “for example”, “in particular”, “such as” or similar expressions.

Where any word or phrase is defined, any other part of speech or other grammatical form of that word or phrase has a cognate meaning.

Where an option is provided, its opposite is also explicitly contemplated. Thus, for example, if the specification states “the stereochemistry may be R”, this would indicate that “the stereochemistry is S” is also contemplated.

As used herein, the term “irreversible” in respect of binding of an inhibitor to an enzyme indicates that the binding can not be reversed without denaturing and/or deactivating the enzyme. This term may refer to mechanistic inactivation.

### DETAILED DESCRIPTION

The present invention relates to the use of N-propargyl-1H-1,2,3-triazoles as inhibitors of nitrification enzymes in soil microorganisms. The inventors have surprisingly found that the presence of the N-propargyl group renders the binding of the inhibitor to the enzyme effectively irreversible, thus extending the inhibitory effect substantially. This in turn may allow farmers to apply nitrogen fertilisers less frequently, leading to substantial cost savings and reduced environmental pollution.

The inventors hypothesise that the presence of the N-propargyl group on the triazole ring may allow for a different binding mechanism to that which operates with other triazoles and which leads to irreversible binding to a nitrification enzyme such as AMO or HAO.

They also hypothesise that if the inhibitor molecule is too lipophilic, the compound cannot penetrate effectively into soil and will therefore be less effective. Accordingly, the compounds used in the present invention are limited to those in which the substituents on the two ring carbon atoms have a combined total of 6 or less carbon atoms and the substituent on the distal end of the alkynyl group is limited to no more than 2 carbon atoms. R² in structure I may be methyl, ethyl, propyl, isopropyl, n-butyl, isobutyl, t-butyl, n-pentyl or a constitutional isomer thereof or n-hexyl or a constitutional isomer thereof. R³ may be H, methyl, ethyl, propyl, isopropyl, n-butyl or a constitutional isomer thereof or n-pentyl or a constitutional isomer thereof, provided that R² and R³ have 1, 2, 3, 4, 5 or 6 carbon atoms in total. Thus, m may be 0, 1, 2, 3, 4 or 5 and n may be 1, 2, 3, 4, 5 or 6, provided that the total of m+n is no more than 6. Where optical isomers are possible, both R and S forms are encompassed. Where steroisomers are possible, all possible stereoisomers (e.g. enantiomers, diastereomers) are encompassed.

The compounds used in the invention may be solids, e.g. crystalline solids, or may be oils or liquids. They may exist as solvates, e.g. hydrates, and these are encompassed by the present invention. They may also exist as salts, such as halide or phosphate salts or salts of an organic acid such as acetic acid or glycolic acid. Once again, such salts are encompassed by the present invention.

In some instances, mixtures of inhibitors may be used. In such instances, at least one, optionally each, of the inhibitors may be an N-propargyl-1,2,3-triazole as described herein.

Triazole-type nitrification inhibitors and methods of using them have been described in WO2021/042169 and the entire contents of that specification are incorporated herein by reference. The compounds used in the present invention, however, are characterised by the presence of an alkynyl group joined to a ring nitrogen of the triazole group by a methylene spacer. The compounds used in the present invention may be made by a general procedure described in Clark et al. (P. R. Clark, G. D. Williams et al. A Scalable Metal-, Azide-, and Halogen-Free Method for the Preparation of Triazoles, Angew. Chem. Int. Ed. 2020, 59, 6740-6744.), the entire contents of which are incorporated herein by reference.

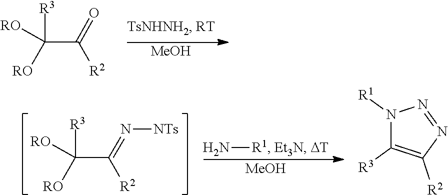

A benefit of this method over previous methods for synthesizing N-substituted 1H-1,2,3-triazoles is that it is tolerant of a wide range of functional groups. In particular, high yields may be obtained for R¹ containing an alkynyl moiety, so as to produce the compounds used in the present invention.

The inhibition of nitrification may be effective at temperatures between about 0 and about 50° C., or between about 0 and 25, 0 and 10, 10 and 50, 20 and 50 or 20 and 40° C., e.g. about 0, 5, 10, 15, 20, 25, 30, 35, 40, 45 or 50° C., or at temperatures above or below these ranges.

Formulations and compositions according to the present invention may be powders, gels, granules, tablets, emulsions, emulsifiable concentrates, microemulsions dispersions or other suitable forms. They may comprise one or more suitable adjuvants e.g. mineral, vegetable or animal oils, solvents (including organic solvents), surfactants etc.

Suitable surfactants include emulsifiers, wetting agents, suspending agents, spreading agents etc. They may be anionic or cationic, non-ionic or zwitterionic. Mixtures of surfactants may be used.

Other adjuvants that may be present include crystallisation inhibitors, viscosity modifiers, dyes, antioxidants, light absorbers, antifoams, complexing agents, pH modifiers, pH buffers, micronutrients, lubricants, dispersants, microbiocides etc.

The compositions may comprise an oil, e.g. a mineral oil or a vegetable oil. Suitable oils include soybean oil, olive oil, rapeseed oil, liquid hydrocarbons, C₈-C₁₈ fatty acids, esters thereof, etc. Mixtures of oils may also be used. The oil may be present from about 0.01 to about 10% of a formulation or composition on a weight basis, or about 0.1 to 10, 1 to 10, 5 to 10, 0.01 to 5, 0.01 to 1, 0.01 to 0.1, 0.1 to 1 or 1 to 5%, e.g. about 0.01, 0.05, 0.1, 0.5, 1, 2, 3, 4, 5, 6, 7, 8, 9 or 10%.

Compositions used in the present invention may comprise from about 1 to about 99% by weight of the triazole compound of Formula (I), described herein, or about 1 to 50, 1 to 20, 1 to 10, 10 to 99, 50 to 99, 10 to 50, 10 to 20 or 10 to 50% thereof, e.g. about 1, 2, 3, 4, 5, 10, 15, 20, 25, 30, 35, 40, 45, 50, 60, 70, 80, 90, 95 or 99%. They may comprise from about 1 to about 99% by weight of one or more adjuvants, or about 1 to 50, 1 to 20, 1 to 10, 10 to 99, 50 to 99, 10 to 50, 10 to 20 or 10 to 50% thereof, e.g. about 1, 2, 3, 4, 5, 10, 15, 20, 25, 30, 35, 40, 45, 50, 60, 70, 80, 90, 95 or 99%. They may comprise from 0 to about 25% by weight of one or more surfactants, or from about 0 to 10, 0 to 5, 0 to 1, 1 to 25, 5 to 25, 10 to 25, 5 to 20 or 10 to 20%, e.g. about 0, 1, 2, 3, 4, 5, 10, 15, 20 or 25% thereof.

The inhibitors of the present invention may be applied in controlled release or sustained release formulations/compositions. They may be microencapsulated or may be embedded in a matrix. The microcapsules may be from about 0.1 to about 500 microns in diameter, or about 1 to 500, 10 to 500, 100 to 500, 200 to 500, 0.1 to 100, 0.1 to 10, 0.1 to 1, 1 to 200, 1 to 100 or 10 to 100 microns, e.g. about 0.1, 0.5, 1, 2, 5, 10, 20, 50, 100, 150, 200, 250, 300, 350, 400, 450 or 500 microns in diameter. The encapsulant may be inorganic or may be organic. It may be polymeric. Suitable matrices or carriers include organic matrices such as wood flour lignin, etc., and inorganic matrices such as silica, clays, titanium dioxide and calcium carbonate. The matrix may be one which dissolves or degrades over time so as to effect sustained release. The skilled person will readily appreciate the range of carriers and/or matrices which may be used.

The inhibitors of the present invention may be applied directly, commonly in solution or in suspension, or as part of a fertiliser composition. Thus, they may be dissolved, suspended or emulsified in a suitable carrier and then applied to the soil. Alternatively or additionally they may be combined with a fertiliser and optionally other components and then applied to the soil. Suitable carriers may be aqueous or may be non-aqueous, e.g. organic. They may in some instances contain no organic solvents. Suitable fertilisers include nitrogen-based fertilisers, e.g. urea-based or ammonia-based fertilisers. They may be organic or may be inorganic. Examples of inorganic fertilisers that may be used include NPK fertilisers, ammonium phosphate, ammonium sulfate, ammonium nitrate, ammonium sulfate nitrate and calcium ammonium nitrate, as well as mixtures of any two or more of these. The fertilisers may comprise animal waste. The fertilisers may be applied as solid fertilisers, e.g. powdered or in pellet form, or as liquid fertilisers. When applied in a fertiliser composition, the inhibitor may be present at a concentration of at least about 0.5 mol % in the fertiliser composition, or at least about 1, 1.5 or 2 mol %, or at a composition of about 0.5 to 5 mol %, 1 to 5, 2 to 5, 0.5 to 2, 0.5 to 1 or 1 to 3 mol %, e.g. about 0.5, 1, 1.5, 2, 2.5 3 3.5 4 4.5 or 5 mol %, or more than 5 mol %.

The inhibitor may be applied in conjunction with a urease inhibitor, such as N-(n-butyl) thiophosphoric triamide (NBPT).

The triazole compounds described herein may be applied no more than once in any 2-week period, or in any 3, 4, 5, 6, 7, 8, 9 or 10 week period. They may be applied once every 2 weeks, or once every 3, 4, 5, 6, 7, 8, 9, 10, 15 or 20 weeks. They may be applied to soil in which a crop is, or is intended to be, grown. They may be applied to said soil once in each growing period of the crop, or twice, three times, or four times in each growing period. The soil to which the triazole compounds are applied may be acidic. They may be mildly acidic or may be strongly acidic. They may have a pH of from about 6.6 to about 4.5 or about 6.5 to 5.5, 5.5 to 4.5, 6.5 to 6, 6 to 5.5, 5.5 to 5, 5 to 4.5 or 5 to 6, e.g. about 4.5, 5, 5.5, 6 or 6.5. Alternatively, they may be non-acidic, e.g. neutral, alkaline, mildly alkaline or strongly alkaline.

It will be understood by the skilled person that the specific compounds, ranges, ratios etc. provided herein are by way of guidance only and in particular cases may vary from those stated herein.

### EXAMPLES

### Material and Methods

All chemicals for the synthesis of the triazole were supplied by Sigma Aldrich Australia. Na₂S₂O₄ was purchased from Chem-Supply. N. europaea (ATCC19718) was purchased from the American Type Culture Collection. N. multiformis was isolated from an aquarium kit (Aquasonic™, BIO-NATURE STANDARD) and its purity was confirmed via 16S rRNA gene sequencing.

Growing Nitrosomonas europaea

Ammonia Oxidising Bacteria (AOB) were cultivated for 3-5 days in Duran glass bottles containing 600 mL of mineral salts media (MSM, pH 7) at 100 rpm at 30° C. in the dark. The slightly loose cap was sealed with an O₂ permeable membrane to ensure aeration (Breathe-Easy® sealing membrane, Sigma Aldrich). The MSM media contains the main bulk medium and consisted of K₂HPO₄ (2.27 g L⁻¹), KH₂PO₄ (0.95 g L⁻¹) and (NH₄)₂SO₄ (0.67 g L⁻¹). The pH was adjusted to 7.0, appropriate for growing AOB. To 1 L of this bulk medium 2 mL of a filter sterilised (0.2 μm Millipore filter) solution of metals was added (Na₂EDTA (6.37 g L⁻¹), ZnSO₄·7H₂O (1.0 g L⁻¹), CaCl₂·2H₂O (0.5 g L⁻¹), FeSO₄·7H₂O (2.5 g L⁻¹), NaMoO₄·2H₂O (0.1 g L⁻¹), CuSO₄·5H₂O (0.1 g L⁻¹), CoCl₂·6H₂O (0.2 g L⁻¹), MnSO₄·H₂O (0.52 g L⁻¹) and MgSO₄·7H₂O (60.0 g L⁻¹)). This media solution was supplemented with 1 v/v % of aqueous Na₂CO₃ (50 g L⁻¹).

### Standard Cell Harvesting Protocol

Cells of N. multiformis and N. europaea were cultivated to the mid-exponential growth phase (OD₆₀₀=0.1, [NO₂⁻] approximately 800 μM) and harvested by filtration onto 0.2 μm membrane filters (Mixed Cellulose Ester (MCE), Sigma Aldrich). The cells were washed with 2×100 mL of 0.1 M sodium phosphate buffer (NaPB), containing 0.2 mM MgSO₄ at pH 7.5. The cells were washed from the filter paper by transferring it to a sterile 50 mL tube and resuspending in NaPB (15 mL), followed by 5 s of vortexing (Ratek, Australia) and 3 s of sonication (Vevor, Australia). The inoculum had an OD₆₀₀ of 1.2-0.9, which was diluted to OD₆₀₀=0.03 and stored at 4° C. until used for the assay (cells can be stored for up to 24 hours without losing activity). Experiments were performed in triplicate.

### Standard Nitrification Inhibition Assay Protocol

In a deep 96-well plate (2 mL capacity), 980 μL of the bacterial inoculum (OD₆₀₀=0.03) was added to the inhibitor (10 μL, 30 mmol L⁻¹ in Milli-Q water), the solutions were mixed thoroughly and pre-incubated in the dark for 5 minutes at 100 rpm and 30° C. (NH₄)₂SO₄ (10 μL, 150 mM, from a prepared sterile solution containing 19.8 g L⁻¹ (NH₄)₂SO₄ in Milli-Q water) was then added using a multi-channel pipette. The plate was covered with a with an O₂ permeable membrane to ensure aeration (Breathe-Easy® sealing membrane, Sigma Aldrich) and incubated in a temperature-regulated rotary incubator (Ratek, Australia) in the dark for 60 minutes at 30° C. and 100 rpm. An aliquot of the reaction solution (50 L) was transferred to a 96-well spectrophotometric plate to which 50 L of Griess reagent was added. The colour was allowed to develop for 15 minutes at room temperature, and the absorbance was measured at 540 nm. Each assay was accompanied by control treatments to determine the 0% and 100% NO₂ signal. The percentage inhibition was calculated according to eqn. 1 from the NO₂ production of the cells in NaPB (i) without additive (‘untreated cells’: 0% signal), (ii) with [NH₄⁺]=3 mM (‘uninhibited’ cells: 100% signal) and (iii) with [NH₄⁺]=3 mM and [inhibitor]=0.3 mM and 0.03 mM (10 mol % and 1 mol % of [NH₄⁺], ‘inhibited cells’). Experiments were performed in triplicate.

$$\% Inhibition = [ 1 - \frac{{[ {NO}_{2}^{-} ]}_{inhibited cells} - {[ {NO}_{2}^{-} ]}_{untreated cells}}{{[ {NO}_{2}^{-} ]}_{uninhibited cells} - {[ {NO}_{2}^{-} ]}_{untreated cells}} ] \times 100 \qquad ( eqn . 1 )$$

### O₂ Consumption Measurements

O₂ consumption rates of cell suspensions of N. europaea were measured using a Clark-type oxygen electrode (Rank Brothers, Cambridge, UK) mounted in a water-jacketed electrode chamber (3 mL capacity) that was connected to a recirculating cooler (Lauda, Austria). The data were recorded using a Data-trax™ (World Precision Instruments, UK) sensor data collection system. All measurements were taken at 20° C. and 1 mL final reaction solution volume. The polarizing voltage was set to 0.6 V. To calibrate the oxygen signal, an excess (approximately 50 mg) of Na₂S₂O₄ was added to 1 mL of Milli-Q water to chemically remove dissolved O₂. Additional O₂ flux was prevented by applying a stopper, and the residual voltage was referred to as “0% O₂”. The voltage at saturated O₂ concentration (“100% O₂”) was determined by measuring the voltage of the equilibrated aerated reaction system consisting of 1 mL Milli-Q water. Sample measurements were taken as follows: The 1 mL reaction mixture, composed of 980 μL N. europaea cell solution in NaPB (OD₆₀₀=0.8; corresponding to approximately 468 μg/L protein) was equilibrated for 5 min in the chamber until the voltage reading was stable. The reaction was then initiated by the addition of (NH₄)₂SO₄ (10 μL of an aqueous 150 mM stock solution, the final concentration in the reaction solution was 3 mM) and the chamber immediately sealed with a stopper. After 4 min of oxygen consumption (a linear rate coefficient of approximately k_{obs}=k=274.7+20.0 nmol O₂ L⁻¹ s⁻¹ was determined), 10 μL of the inhibitor stock solution of MPT in DMSO (1.2 mM and 0.6 mM) were added via a 10-μL Eppendorf pipette through a capillary opening, ensuring the emergence of the pipette tip in the solution. The voltage was recorded over a period of 25 min in intervals of 5 s. The trace describing the O₂ concentration after addition of (NH₄)₂SO₄ against the time (initial 15-140 s) was used as the baseline O₂ consumption for the uninhibited cells, whereas the trace describing the consumption in the presence of the inhibitor (255-1740 s time window) was used to determine the rate of O₂ consumption in the presence of inhibitor. All experiments were conducted in triplicate at 20° C. under constant stirring. The voltage was converted to [O₂] according to equation 2:

$${[ {O}_{2} ]}_{t} = \frac{voltage {( sample )}_{t} - voltage ( 0 \% {O}_{2} )}{voltage ( 100 \% {O}_{2} ) - voltage ( 0 \% {O}_{2} )} \times 280 μM \qquad ( eqn . 2 )$$

Kinetic parameters were determined via GraphPad Prism software, using simple linear regression for uninhibited cells and non-linear fit (curve fit) first order decay with an R²>0.9 for inhibited cells.

### Activity Recovery Assay

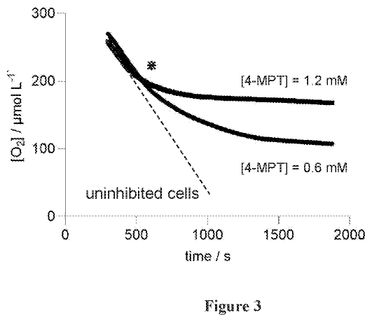

Cells were harvested using the ‘standard cell harvesting protocol’. The bacteria solution was adjusted to an OD₆₀₀=0.8 (100 μg/L protein) and 980 μL aliquots were transferred to 1.5 mL centrifuge tubes (Eppendorf®, polypropylene). 10 μL of the inhibitor solution (150 mM in DMSO) was added and pre-equilibrated for 5 min; 10 μL of aqueous 150 mM (NH₄)₂SO₄ (final concentration in tube=3 mM) was added by via multichannel pipette to ensure simultaneous addition in each tube. The tubes were incubated in a temperature-regulated rotary incubator (Ratek, Australia) for 30 min at 30° C. at 100 rpm in the dark. After 30 minutes, a 50 μL aliquot was transferred to a 96-well plate (Greiner Cellstar®, polystyrene), 50 μL of Griess reagent was added and the mixture incubated for 15 minutes. The absorbance was measured photometrically (Clariostar® BMG Labtech, Australia) at 540 nm. The cells were washed repeatedly (3×) by periodic centrifuging (Boeco, Germany; 10.000 rpm, 10 min) and resuspending the pellet in 1 mL NaPB. After the final centrifuging step, the pellet was resuspended in 990 μL NaPB and re-incubated with (NH₄)₂SO₄ (10 μL of an aqueous 150 mM solution), and the NO₂ concentration was measured under the previously described conditions. Each assay was accompanied by control treatments to determine the 0% and 100% NO₂ signal. The percentage activity was normalised according to the NO₂ production of the cells in NaPB (i) without additive (‘untreated cells’: 0% signal), with [NH₄⁺]=3 mM (‘uninhibited’ cells: 100% signal) and (ii) with [NH₄⁺]=3 mM and [inhibitor]=1.5 mM. Experiments were performed in triplicate. Results are shown in FIG. 2.

### Acute Toxicity Test

Cells were harvested using the ‘standard cell harvesting protocol’. The bacteria solution was adjusted to an OD₆₀₀=0.8 (100 μg/L protein). The solution was divided into 1 mL aliquots and transferred to a 24-well tissue culture plate (Greiner Cellstar®, polystyrene Tissue Culture treated). Each well contained 980 μL of bacterial solution, 10 μL of a 150 mM aqueous (NH₄)₂SO₄ solution and 10 μL of a 150 mM inhibitor solution. The well plate was sealed with an 02 permeable membrane to ensure aeration (Breathe-Easy® sealing membrane, Sigma Aldrich) and incubated in the dark for 4 h at 30° C. and 100 rpm. After incubation, cells were transferred into a centrifuge tube and sedimented at 10,000 rpm for 10 minutes. The supernatant was discarded, and cells were re-suspended in 1 mL NaPB. A 5 μL aliquot was transferred into a 96-well plate and the bacterial stain (LIVE/DEAD™ BacLight™ Bacterial Viability Kit for microscopy, ThermoFisher Scientific) was added following the manufacturer's guideline. 10 μL of the solution was transferred onto a microscopic slide (Fisher Scientific, Australia, microscope slides 7.6 cm×2.5 cm (L×W), thickness 1-1.2 mm). Ten images were taken per treatment with a fluorescence microscope (Leica DM6000, Germany) using the red filter setting (excitation:

$$\% Live = \frac{Live cell count}{total cell count} \times 100 \qquad ( eqn . 3 )$$

$$\% Dead = \frac{Dead cell count}{total cell count} \times 100 \qquad ( eqn . 4 )$$

575/30 nm (dichromatic) DC: 600; emission: 635/40 nm) and green channel using filter setting (excitation: 500/20 nm DC: 515; emission: 535/30 nm) to detect dead and live cells respectively. The percentage of live and dead cells was calculated via eqns. 3 and 4:

### Soil Incubation Studies to Determine Soil Mineral N-Transformations

Soils used in these studies were air-dried, ground and sieved (2 mm) prior to use. Soil microcosm incubations were carried out in 250 mL polypropylene specimen containers (Sarstedt, Germany), containing 20 g oven dry-weight equivalent of soil. Microcosms were treated with half the volume of water required to meet the desired water-filled pore space (WFPS %) of 60% and pre-incubated at 25° C. for seven days to revive soil microbial activity. Following pre-incubation, the remaining volume to reach the WFPS % was applied as one of the treatment solutions: (1) fertiliser ((NH₄)₂SO₄,) 100 mg kg⁻¹, (2) fertiliser+0.5 mol % 4-MPT, (3) fertiliser+2.5 mol % 4-MPT, (4) fertiliser+5 mol % 4-MPT or (5) fertiliser+5 mol % compound 1, with three replicates of each treatment per time interval (n=3). Throughout the incubation period soil microcosms were kept aerated by removing the lid for 10 minutes every 2-3 days to allow gas exchange, and moisture levels were replenished via addition of Milli-Q water as required. At the end of the desired incubation period (i.e., after 0, 3, 7, 14, and 21 days, respectively), soil microcosms were removed and destructively sampled by treating with 2 M aqueous potassium chloride solution (KCl, 100 mL) and shaking for 1 h. Soil-KCl solutions were filtered (Whatman No. 42), and the filtrates were stored at −20° C. until the conclusion of the experiment, when all KCl extracts were analysed for the concentration of soil mineral nitrogen from ammonium (NH₄⁺—N) and from nitrate (NO₃⁻—N; the conversion of NO₂ to NO₃⁻ in soils is very rapid) after appropriate dilutions using Segmented Flow Analysis (San++, Skalar, Breda, The Netherlands). Results are reported as the mean of three replicates, errors reported are standard errors of the mean. Errors associated with raw data were carried through calculations using standard error propagation protocols.

### Soil Incubation Studies to Determine N₂O Production

Soil incubations were performed in three replicates. Each sample consisted of 6 g of sieved soil (2 mm). The soil was air-dried after collection and pre-incubated prior to the experiment for seven days at 50% water holding capacity (WHC). The soil was transferred to a gas chromatography vial (22 mL volume, clear glass, Macherey-Nagel, Germany) and compacted densely to allow 5.5-6.2 cm headspace. The soil in the vials was incubated for a total of 21 days at a constant temperature of 21° C. with an open lid to ensure gas circulation. The control incubations contained ‘untreated’ soil (deionized H₂O only) and ‘fertiliser only’ soil ((NH₄)₂SO₄; 50 mg kg⁻¹ soil) and accompanied each measurement. (NH₄)₂SO₄ was applied as an aqueous solution (1 g NH₄⁺ mL⁻¹ H₂O) and introduced on the soil surface to mimic field conditions. Three inhibitor solutions were prepared from DMP and 4-MPT at concentrations of 0.5 mol %, 2.5 mol % and 5 mol % of applied fertiliser-N and applied on the soil surface. The soils reached 60% WHC after the treatments had been applied and were kept at that moisture level by periodically adding deionized water to compensate for evaporation losses. At the day of measurement (day 1, 3, 5, 7, 14 and 21 after fertilization) the vials were closed gas-tight with a rubber septum and aluminium lid and opened again after each measurement. The N₂O emission was analysed using a gas chromatograph equipped with an electron capture detector and flame ionization detector (GC-ECD/FID; Clarus 580, Perkin Elmer). A linear regression slope was used to calculate N₂O—N production rates, F, according to eqn. 5:

$$F = \frac{\frac{Δ C}{Δ t} V {T}_{0} M}{m {T}_{a} {V}_{m}} \qquad ( eqn . 5 )$$

where ΔC/Δt represents the change of the N₂O concentration over the time interval t in ppbv, V represents the headspace volume in L, M represents the molar mass of N in N₂O, m represents the amount of soil in g dry weight, Vₘ represents the molar volume of ideal gases (22.414 L mol⁻¹) at 0° C. and 101.325 kPa, corrected for the gas sampler using T₀ (273.15 K) and Ta (air temperature in K).

### Statistics

Statistical analysis was performed through Minitab 18 using P<0.05 as the level of statistical significance. All results were reported as mean±standard error of the mean. Significances among three treatments were compared by the least significance differences P<0.05 level using one-way ANOVA.

Synthesis of Substituted 1,2,3-triazoles

Reaction progress was monitored by mass spectroscopy (HRMS, Thermo, Bremen, Germany) operated in positive mode. Purification by flash column chromatography was performed using Divisil Chromatographic Silica Media LC60A 40-63 micron with ethyl acetate (Chem Supply, Australia) and petroleum spirit (Thermo Fisher, Australia). All ¹H NMR and ¹³C NMR and spectra were recorded on a 500 MHz Bruker spectrometer (500 or 126 MHz, respectively) using the reported solvent resonance as the internal standard. Chemical shifts are given in parts per million (ppm) with the splitting patterns indicated as follows: s, singlet; d, doublet; t, triplet; q, quartet; p, pentet; m, multiplet; dd, doublet of doublets. The coupling constant J is reported in Hertz (Hz). Inhibitors N001, N003, N013, N014, N018, N023, and Compound 2, Compound 3 and Compound 4 were kindly provided by Bethany Taggert, University of Melbourne.

### General Procedure:

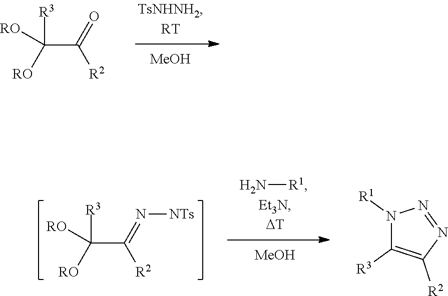

The reactions were performed in a round-bottom flask for larger quantities (1 g and above) with or without heating as required and smaller quantities were synthesised in a microwave tube (Biotage®, Sweden). The required α-ketoacetal (1.05 equiv.) was dissolved in methanol (resulting in a 0.4 M solution) in a reaction vial. 4-Methylbenzenesulfonohydrazide (tosylhydrazide, TsNHNH₂) (1.0 equiv.) was added in portions and the reaction stirred at room temperature for several minutes until the complete transformation of the starting material to the hydrazone was confirmed by ESI-MS. The primary amine (1.1 equiv.) and triethyl amine (1.1 equiv.) were then added to the reaction mixture. Reactions above the boiling point of methanol and the primary amine were performed in sealed Biotage® microwave tubes and exposed to microwave irradiation (Biotage® Initiator+, Sweden) or heated in a fume cupboard under reflux under an inert atmosphere. After the required reaction time the reactions were cooled to room temperature and concentrated in vacuo (Buechi, Germany). The crude oil was dissolved in dichloromethane (DCM):water (1:1), and the aqueous layer was extracted three additional times with DCM. The combined organic layers were washed with brine, dried over Na₂SO₄, concentrated in vacuo and purified via flash column chromatography if required.

### 4-Methyl-1-(prop-2-yn-1yl)-1H-1,2,3-triazole (4-MPT)

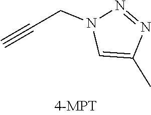

Following the General Procedure, 1,1-dimethoxypropan-2-one (250 mg, 2.20 mmol) in methanol (5 mL) with tosylhydrazide (388 mg, 2.08 mmol) were reacted in a 10 mL microwave tube (Biotage®, Sweden). After complete consumption of the starting material, propargylamine (129 mg, 2.29 mmol) and triethylamine (232 mg, 2.30 mmol) were added and the tube sealed. After 20 minutes at 120° C. and cooling to room temperature, the reaction mixture was concentrated in vacuo to give an orange oil. The crude oil was dissolved in DCM:water (1:1), and the aqueous layer was extracted additional three times with DCM. The combined organic fractions were washed with brine, dried over Na₂SO₄, concentrated in vacuo and purified via flash column chromatography (ethylacetate:petroleum spirits (1:1)) to yield 4-MPT as an orange oil (85%). The oil was cooled overnight at 4° C. to yield 4-MPT as a colourless crystalline solid.

4-MPT (crude orange oil): ¹H NMR (500 MHz, CDCl₃) δ 7.50 (d, J=1.0 Hz, 1H, triazole-H), 5.11 (d, J=2.6 Hz, 2H, aliphatic-H), 2.54 (t, J=2.6 Hz, 1H, alkyne-H), 2.34 (d, J=0.9 Hz, 3H, aliphatic-H).

4-MPT (crude orange oil): ¹³C NMR (126 MHz, CDCl₃) δ 143.8 (triazole-C), 120.9 (triazole-C), 76.1 (broad, 2C, alkyne-C), 39.6 (aliphatic-C), 10.8 (aliphatic-C).

ESI-HRMS Calculated for C₆H₈N₃⁺ ([M+H]⁺): m/z 122.0713; found m/z 122.0713.

4-MPT (crystalline): ¹H NMR (500 MHz, CDCl₃) δ 7.49 (s, 1H, triazole-H), 5.10 (d, J=2.6 Hz, 2H, aliphatic-H), 2.53 (t, J=2.6 Hz, 1H, alkyne-H), 2.33 (s, 3H, aliphatic-H).

4-MPT (crystalline): ¹³C NMR (126 MHz, CDCl₃) δ 143.8 (triazole-C), 120.9 (triazole-C), 76.1 (broad, 2C, alkyne-C), 39.6 (aliphatic-C), 10.8 (aliphatic-C).

Crystal structure of 4-MPT.

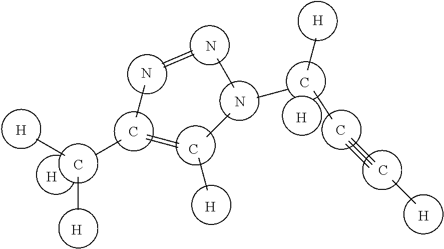

TABLE 1
Crystallographic data for 4-MPT.^{[a]}

| Empirical formula | C₆H₇N₃ |
|---|---|
| Formula weight | 121.15 |
| Temperature/K | 100.0(1) |
| Crystal system | orthorhombic |
| Space group | P2₁2₁2₁ |
| a/Å | 4.3363(2) |
| b/Å | 10.1113(5) |
| c/Å | 14.8926(9) |
| α/° | 90 |
| β/° | 90 |
| γ/° | 90 |
| Volume/Å³ | 652.98(6) |
| Z | 4 |
| P_{calc} g/cm³ | 1.232 |
| μ/mm⁻¹ | 0.651 |
| F(000) | 256.0 |
| Crystal size/mm³ | 0.075 × 0.045 × 0.019 |
| Radiation | Cu Kα (λ = 1.54184) |
| 2Θrange fir data collection/° | 10.576 to 158.242 |
| Index ranges | −5 ≤ h ≤ 5, −12 ≤ k ≤ 9, −18 ≤ |
| | l ≤ 18, |
| Reflections collected | 6438 |
| Independent reflections | 1381 [Rᵢₙₜ = 0.0783, R_{sigma} = 0.0550] |
| Data/restraints/parameters | 1381/0/83 |
| Goodness-of-fit on F² | 1.121 |
| Final R indexes [I >= 2σ (I)] | R₁ = 0.0560, wR₂ = 0.1437 |
| Final R indexes [all data] | R₁ = 0.0598, wR₂ = 0.1462 |
| Largest diff. peak/hole/e Å⁻³ | 0.26/−0.36 |
| Flack parameter | 0.3(6) |

^{[a]}Data was collected on a Rigaku XtaLAB Synergy-S Dual Microfocus X-ray diffractometer using Cu—Kα radiation (λ = 1.54184) at 100 K. Crystals were transferred directly from the flask into a cryoprotective oil and then immediately mounted on the diffractometer at the data collection temperature to prevent solvent loss. The data was processed using CrysAlisPro. The structure was solved using the intrinsic phasing routine in SHELXT and refined using a fullmatrix least square procedure based upon F2 using SHELXL within OLEX2. For all structures, the positions of all non-hydrogen atoms were refined using anisotropic displacement parameters. Hydrogen atoms were placed geometrically, and their positions were constrained to geometrical estimates using the riding model.

### 4-Methyl-1-(n-propyl)-1H-1,2,3-triazole (Compound-1)

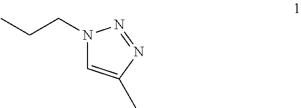

Following the General Procedure, 1,1-dimethoxypropan-2-one (250 mg, 2.20 mmol,) in methanol (5 mL) and tosylhydrazide (388 mg, 2.08 mmol) were reacted in a 10 mL microwave tube (Biotage®, Sweden). After complete consumption of the starting material, n-propylamine (129 mg, 2.29 mmol) and triethylamine (232 mg, 2.30 mmol) were added and the tube sealed. After 20 minutes at 120° C. and cooling to room temperature, the reaction mixture was concentrated in vacuo to an orange oil. The crude oil was dissolved in dichloromethane:water (1:1) and the aqueous layer was extracted additional three times with dichloromethane. The combined organic fractions were washed with brine, dried over Na₂SO₄ and concentrated in vacuo to yield 1 as a yellow oil (72%).

¹H NMR (500 MHz, CDCl₃) δ 7.23 (s, 1H, triazole-H), 4.22 (t, J=7.1 Hz, 2H, aliphatic-H), 2.29 (d, 3H, aliphatic-H), 1.86 (m, 2H, aliphatic-H), 0.89 (t, J=7.4 Hz, 3H, aliphatic-H).

¹³C {¹H} NMR (126 MHz, CDCl₃) δ 142.8 (triazole-C), 118.7 (triazole-C), 59.9 (aliphatic-C), 33.6 (aliphatic-C), 25.2 (aliphatic-C), 10.9 (aliphatic-C).

ESI-HRMS Calculated for ([M+H]⁺): m/z 126.1026; found m/z 126.1026.

### Results and Discussion

The Compound 4-methyl-1-(prop-2-yn-1yl)-1H-1,2,3-triazole (4-MPT)

4-MPT is a 1-alkynyl-4-alkyl substituted 1,2,3-triazole. It shows significant inhibition towards the ammonia oxidising bacteria N. multiformis and N. europaea of 75% and 77%, respectively.

Sensitivity of Ammonia Oxidising Bacteria (AOB) Against 1,4-disubstituted 1,2,3-triazoles
a) Non-Alkynyl Group Containing 1,4-disubstituted 1,2,3-triazoles

Previously, compounds containing various substituents (with or without additional functional groups) were tested in 28-day soil incubation studies. Their activity has not been shown in pure bacterial cultures of AOB. The inventors tested herein their percent inhibitory effect against pure cultures of two AOB strains of N. multiformis and N. europaea. The reported compounds contain an alkyl group in R² position (with reference to the structure in Table 2) and varying functional groups in R¹ including an (I) aliphatic chain; (II) ester functional group; (III) amine functional group; (IV) alkoxy functional group; and (V) alkene functional group.

The percent inhibition assay was prepared and analysed as described earlier.

TABLE 2
Substitution pattern of 1,4-disubstituted 1,2,3-triazoles containing varying functional
groups tested following the ‘standard nitrification inhibition assay protocol’.

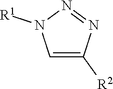

| Com- | | | Functional | |
|---|---|---|---|---|
| pound | R¹ | R² | group | Properties |
| N001 | n-C₅H₁₁ | n-C₄H₉ | none | non-polar |
| N003 | n-C₄H₉ | CH₂C(═O)OC₂H₅ | ester | Non-polar; polar aprotic |
| N006 | n-C₄H₉ | C₃H₆NH₂ | amine | Non-polar; polar protic |
| N014 | n-C₄H₉ | C₂H₄OCH₃ | alkoxy | Polar protic; polar aprotic |
| N018 | n-C₃H₇ | CH₂HC═CH₂ | alkene | non-polar |
| N023 | CH₂C(═O)OC₂H₅ | n-C₃H₇ | ester | Non-polar; polar aprotic |

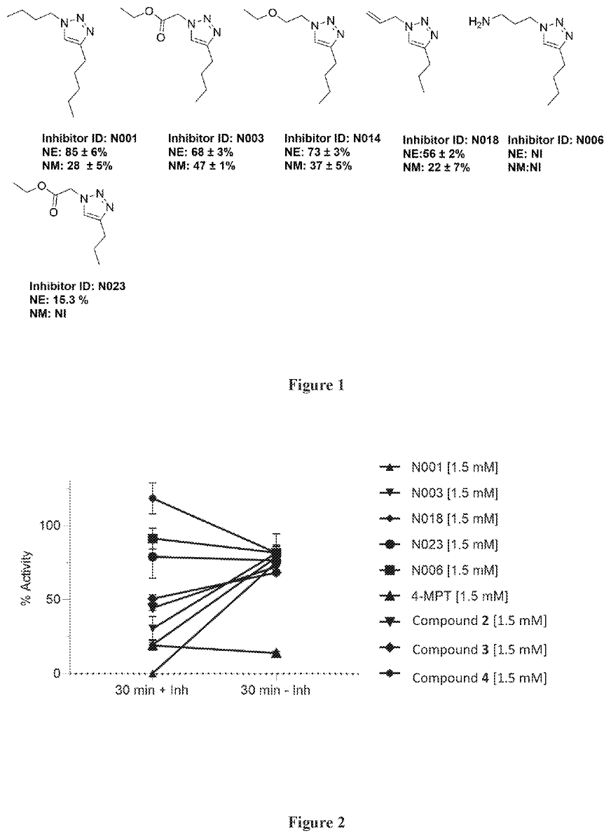

FIG. 1 shows the structure-activity relationship (SAR) of 1,4-disubstituted 1,2,3-triazoles, determined using N. europaea (NE) and N. multiformis (NM) by monitoring the NO₂ production following the standard nitrification inhibition assay protocol. The incubations in NaPB with 1 v/v % DMSO were performed at pH=7.5 and 30° C. with [NH₄⁺]=3 mM, [inhibitor]=0.3 mM (10 mol % of [NH₄⁺]) at 100 rpm for 60 minutes in the dark. Standard errors were determined from three biological and three technical replicates. No inhibition is indicated as NI.
b) Alkynyl Group Containing 1,4-disubstituted 1,2,3-triazoles

In this experiment the importance of the alkynyl group was investigated through SAR studies of reported 1,4-disubstituted 1,2,3-triazoles containing the alkynyl group in the 1 position.

TABLE 3
Percent inhibition of N. europaea (NE) and N. multiformis (NM) by 1-alkynyl
substituted 1,2,3-triazoles. The NO₂⁻ production was determined according to the ‘standard
nitrification inhibition assay protocol’. The incubations in NaPB with 1 v/v % DMSO were
performed at pH = 7.5 and 30° C. with [NH₄⁺] = 3 mM, [inhibitor] = 0.3 mM (10 mol % of
[NH₄⁺]) at 100 rpm for 60 min in the dark. Standard errors were determined from three
biological and three technical replicates. No inhibition is indicated as NI.

| Compound | Structure | % Inhibition NE | % Inhibition NM |
|---|---|---|---|
| 2 | 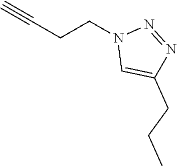 | 21 ± 2 | 40 ± 1 |
| 3 | 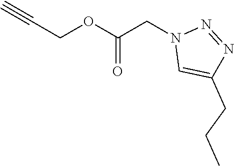 | 49 ± 2 | 47 ± 2 |
| 4 | 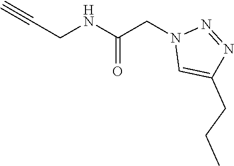 | 7.1 ± 1 | NI |

The inhibitory effect fluctuates between all three compounds. Whilst compound 3 has the highest activity, the alkynylalkyl substituted compound 2 has a significantly reduced efficacy with both bacterial strains. This shows that the presence of an alkenyl substituent anywhere in the 1-position does not necessarily create a potent inhibitor.

### Effect of 4-MPT on Ammonia Monooxygenase and Hydroxylamine Oxireductase

AOB oxidises NH₃ to NO₂⁻. The oxidation is carried out by the two enzymes ammonia monooxygenase (AMO) and hydroxylamine oxireductase (HAO). N. europaea and N. multiformis contain naturally both enzymes. To identify the affected enzyme, the NH₃ is supplemented by NH₂OH, the substrate of the second enzyme.

TABLE 4
Effect of 4-MPT on hydroxylamine oxireductase (HOA). The test was
performed on N. europaea (NE) and N. multiformis (NM) for the
NO₂⁻ production following the ‘standard nitrification inhibition
assay protocol’. The incubations in NaPB with 1 v/v % DMSO were
performed at pH = 7.5 and 30° C. with [NH₄⁺] = 3 mM
or [NH₂OH] = 3 mM or, [inhibitor] = 0.3 mM
(10 mol % of [NH₄⁺] and [NH₂OH]) at 100
rpm for 60 min in the dark. Standard errors were determined
from three biological and three technical replicates.

| Substrate | % Inhibition NE | % Inhibition NM |
|---|---|---|
| NH₃ | 77 ± 1 | 75 ± 4 |
| NH₂OH | 28 ± 3 | 33 ± 4 |

The test clearly reveals that the main reason for the reduced NO₂⁻ production is inhibition of AMO. Although HAO also shows a significantly lower NO₂⁻ production in the presence of 4-MPT, the loss of activity could result from the inactivation of AMO. Both enzymes are biochemically connected through an electron shuttle mechanism, where HAO generates an excess of two electrons that are transferred to AMO to fill the gap of the two electrons lost due to NH₃ oxidation and the concomitant reduction of oxygen to produce water as by-product. (D. J. Arp, L. A. Sayavedra-Soto, N. G. Hommes. Molecular biology and biochemistry of ammonia oxidation by Nitrosomonas europaea. Arch. Microbiol. 2002, 178, 250-255.). Thus, it is likely that a disruption of the electron transfer chain due to AMO inhibition, in this case by 4-MPT, leads also to (partial) inhibition of HAO. Given that the inhibition of NO₂⁻ formation is more significant when NH₃ is the substrate, it is suggested that the AMO is indeed the enzyme targeted by 4-MPT.

### Binding Studies

Binding studies were performed following the ‘activity recovery assay’ with the nitrifying bacteria N. europaea and N. multiformis. The initial measurement represents the activity of the cells in the presence of inhibitor. In FIG. 2 the results of the recovery assay are shown. Overall, the reactivity of cells exposed to 1,4-disubstituted 1,2,3-triazoles with various substitution pattern was able to be recovered after three washing steps. The activity of cells exposed to 4-MPT, however, could not be recovered, indicating a mechanism-based inactivator.

### Mechanistic Inhibition

The binding studies have shown that the inhibitory effect is of mechanistic nature. To validate the results, compound 1 was synthesized, which contains instead of the propargyl substituent in 4-MPT an n-propyl substituent. To determine its efficiency as nitrification inhibitor, the percent activity was tested with pure cultures of N. europaea and N. multiformis. The results with both AOB strains show that replacing the alkyne moiety by an alkyl group with the same number of carbon atoms leads to complete loss of inhibitory activity.

TABLE 5
Inhibitory effect of alkyl and alkylalynyl substituted 1,2,3-triazoles.
No inhibition is indicated as NI.

| | | % | % |
|---|---|---|---|
| Com- | | Inhibition | Inhibition |
| pound | Structure | NE | NM |
| 2 | 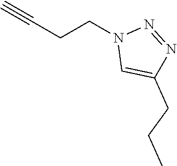 | 21 ± 2 | 40 ± 1 |
| N013 | 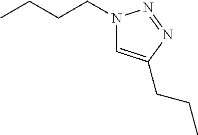 | 60 ± 1 | 54 ± 2 |
| 4-MPT | 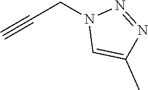 | 77 ± 1 | 75 ± 4 |
| 1 | 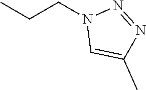 | NI | NI |

In comparison to the prior art, when comparing N013 to compound 2, the removal of the triple bond in N013 increased the activity, supporting the suggestion that these compounds act as different mode inhibitors.

### Oxygen Potential Measurement

Ammonia monooxygenase belongs to the family of metal oxidases, and its oxygen turnover is proportional to the NO₂⁻ production (nitrification). The oxygen concentration was measured over 20 minutes according to ‘O₂ consumption measurements’. After pre-equilibration of the bacterial solution in the electrochemical chamber (0-300 seconds, not shown in Figure [NH₄⁺] (3 mM aqueous solution) was added, initiating the oxidation. In the absence of inhibitor, the O₂ consumption followed zero-order kinetics with an average rate coefficient k=275±20 nmol O₂ L⁻¹ s⁻¹ in the time interval of 300-600 seconds. 4-MPT was added at the 600 second timepoint (indicated with [symbol] ) at two different concentrations of 0.6 mM and 1.2 mM in two separate experiments, which correspond to 20 mol % and 40 mol % with respect to the NH₄⁺ concentration. The rate of oxygen consumption decreased significantly (P<0.005) for both inhibitor concentrations immediately after addition of the inhibitor. The decay profile after the addition of 4-MPT changed to follow first-order kinetics. The observed rate coefficients, k_{obs}, for the O₂ consumption in the presence of MPT were determined as 2.7×10⁻³ s⁻¹ and 4.5×10⁻³ s⁻¹ for [MPT]=0.6 mM and 1.2 mM, respectively. As the rate of O₂ consumption in the presence of 4-MPT is a measure for the rate of enzyme inhibition, the observed increase of the rate of inhibition with increasing [4-MPT] (doubling [4-MPT] increased the rate of inhibition by a factor of about two in our experiment), is indicative for a mechanistic inhibition, where the number of active enzymes declines over time.

### Binding Studies

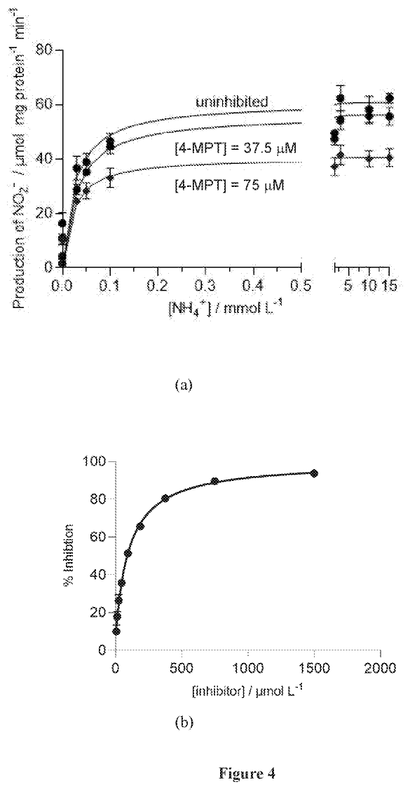

These studies were performed using a modified procedure of that described earlier. In FIG. 4a, the rate of NO₂⁻ production was measured as a function of [NH₄⁺] to determine whether the inhibitor follows Michaelis-Menten kinetics. The [NH₄⁺] was 0.003 mM, 0.03 mM, 0.5 mM, 1.5 mM, 3 mM, 10 mM, 15 mM and 30 mM. [4-MPT] remained constant at 0.3 mM. The Michaelis-Menten plots show saturation kinetics, where the NO₂ production became independent of [NH₄⁺] beyond 0.5 mmol L⁻¹. Furthermore, cells treated with 4-MPT did not lead to an increased NO₂ production even when [NH₄⁺] was increased by up to four orders of magnitude. Determination of the Michaelis-Menten parameters via hyperbolic analysis revealed that the value of Kₘ₍ₐₚₚ₎ was unchanged within experimental error, whereas Vₘₐₓ₍ₐₚₚ₎ decreased with increasing [4-MPT](the suffix ‘app’ indicates that these constants were determined from bacterial cells and not the purified enzyme).

TABLE 6
Michaelis-Menten kinetic parameters of the [NH₄⁺]-
dependent production of NO₂⁻ by N. europaea
in the absence and presence of 4-MPT.^{[a,b]}

| | Concentration/ | Vₘₐₓ₍ₐₚₚ₎/μmol | Kₘ₍ₐₚₚ₎/ |
|---|---|---|---|
| Inhibitor | μM | (mg protein min)⁻¹ | μM |
| none | — | 70.4 ± 7.3 | 29.9 ± 4.9 |
| 4-MPT | 37.5 | 52.7 ± 2.0 | 30.2 ± 0.3 |
| 4-MPT | 75.0 | 39.2 ± 2.0 | 23.7 ± 4.0 |

^{[a]}[4-MPT] was chosen to achieve partial inhibition of the NO₂⁻ production.
^{[b]}The suffix ‘app’ in Vₘₐₓ₍ₐₚₚ₎ and Kₘ₍ₐₚₚ₎ indicates that these constants were determined from bacterial cells and not the purified enzyme. Standard errors were calculated from three biological replicates, each performed with three technical replicates.

Such a behaviour is indicative for a non-competitive inhibition mode, i.e., 4-MPT is not competing with NH₃ for the same binding site in AMO. FIG. 4 b) shows the dose response curve of 4-MPT, where [4-MPT] was 1500 μM, 750 μM, 375 μM, 188 μM, 94 μM, 47 μM, 23 μM, 12 μM and 6 μM. Generally, the percent inhibition increases with the inhibitor concentration, showing that the inhibitor concentration determines the NO₂ production.

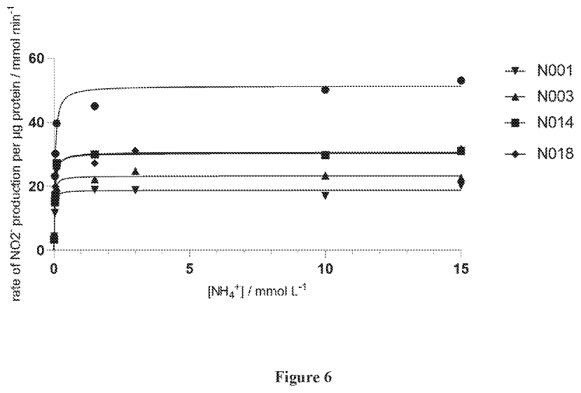

Mode of inhibition studies with remaining inhibitors N001, N003, N014 and N018 were conducted as a proof of concept. Results are shown in FIG. 6. Similar to FIG. 4a with no inhibitor, it can be seen that the NO₂⁻ production initially increases sharply, indicating that these inhibitors are reversible inhibitors, which supports the hypothesis that the alkynyl group in 4-MPT contributes to the irreversible binding to the enzyme.

### Acute Toxicity Assay

A toxicity test was performed following the binding studies. The details are described earlier herein.

TABLE 7
Acute toxicity of 4-MPT with cells of N. europaea. Control
treatment contained cells without inhibitor, both treatments contained
NH₄⁺ to avoid cell death caused by starvation. The incubations
were performed in NaPB with 1 v/v % DMSO at pH = 7.5 and 30°
C. with [NH₄⁺] = 3 mM, [4-MPT] = 1.5 mM (50 mol %
of [NH₄⁺]) at 100 rpm for 12 h in the dark. Standard
errors were determined from ten microscopic images.

| Treatment | % Live | % Dead |
|---|---|---|
| Control | 83 ± 10 | 17 ± 10 |
| 4-MPT | 75 ± 8 | 25 ± 8 |

### Soil Incubation Studies

TABLE 8
Specifications of the soils studied in this work.^{[a]}

| | Soil | Soil | Soil | Soil | Soil |
|---|---|---|---|---|---|
| Parameter | A | B | C | D | E |
| pH (CaCl₂) | 5.9 | 6.3 | 5.5 | 4.7 | 7.5 |
| pH (H₂O) | 6.4 | 6.7 | 6.0 | 5.4 | 8.1 |
| Sand (%) | 42 | 8 | 74 | 87 | 5 |
| Slit (%) | 35 | 77 | 16 | 8 | 78 |
| Clay (%) | 24 | 15 | 10 | 4 | 17 |
| TOC (%) | 4.86 | 1.0 | 2.05 | 0.47 | 0.52 |
| TC (%) | 5.6 | 1.0 | 2.1 | 0.5 | 2.12 |
| TN (%) | 0.43 | 0.15 | 0.04 | 0.11 | 0.05 |
| min-N (NH₄⁺)/mg kg⁻¹ soil | 5.6 | 0.24 | 0.97 | 0.63 | 1.12 |
| min-N (NO₃⁻)/mg kg⁻¹ soil | 70 | 70.9 | 39.7 | 27.7 | 21.8 |
| WHC/g 100 g⁻¹ soil | 78 | 40 | 51 | 28 | 44 |

^{[a]}Mean values from three replicates; TOC = total organic carbon, TC = total carbon, TN = total nitrogen, min-N = mineral-N, WHC—water holding capacity (WHC). Soil A was collected from an agricultural site in Victoria, Australia; soils B-E were collected from agricultural sites in Germany.

Measurement of NH₄⁺ and NO₃⁻ in Soil A

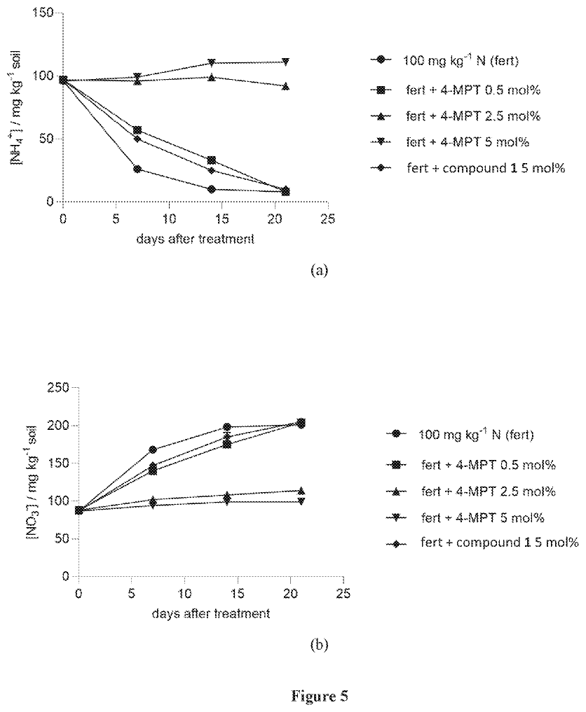

To determine the nitrification inhibitory effect of 4-MPT in vivo, soil incubations were performed by measuring concentration-time profiles for NH₄⁺ and NO₃⁻ in an acidic Australian soil (soil A, pH=5.9) over 21 days. 4-MPT was tested at three different concentrations (0.5 mol %, 2.5 mol % and 5 mol % of applied fertiliser-N) and compared with the alkyl-substituted compound 1 (5 mol % of applied fertiliser-N) to confirm the requirement of the N-propargyl substitutent for inhibitory activity (FIG. 5).

Loss of NH₄⁺ occurred rapidly both without NI and with compound 1, with negligible levels of NH₄⁺ remaining after 21 days of incubation (FIG. 5a) and quantitative conversion to NO₃ (FIG. 5b). Also, while 4-MPT at the lowest application rate noticeably delayed NH₄⁺ loss within the first 15 days, it was not sufficiently effective to retain NH₄⁺ in the soil beyond 21 days. In contrast, at the higher application rates of 2.5 and 5 mol %, 4-MPT enabled to quantitatively suppress NH₄⁺ conversion in the soil over the entire incubation period with a statistical significance of P<0.001 when comparing inhibitor treatments to the control treatment with (NH₄)₂SO₄ alone and when comparing to the 5 mol % treatment with Compound 1. These findings were also reflected by the lack of NO₃ production over the duration of the incubation. In fact, the inhibitory performance of 4-MPT at the 2.5 mol % application rate was only marginally poorer than with the higher application rate of 5 mol % (P<0.01 for day 7 with 2.5 mol % MPT and P<0.001 for 2.5 and 5 mol % 4-MPT on the other days). These data clearly illustrate MPT's superior inhibitory performance, which can be assigned to MPT's distinct mode of inhibition.

### Measurement of the N₂O Production in Soils B-E

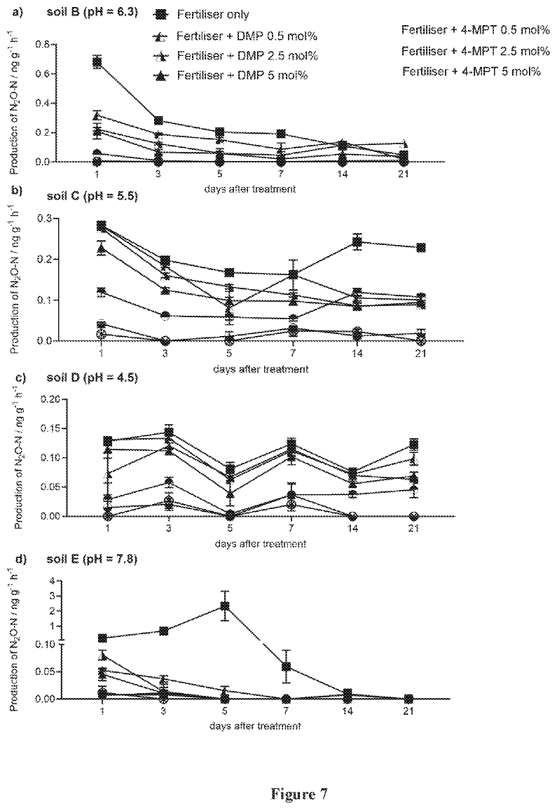

Soil incubations were performed with four German soils (soils B-E) with varying pH, and the N₂O production rates in the presence of 4-MPT were compared with those measured using DMP at the same application rates (i.e., 0.5 mol %, 2.5 mol % and 5 mol % of applied fertiliser-N) as well as in the absence of NI. The results of the soil incubation are shown in FIG. 7 and in Table 9. In the first week after fertiliser application to soil B (pH=6.3), the N₂O production rate by the uninhibited soil was the highest with 0.68 ng g⁻¹ soil h⁻¹ at day 1 and 0.19 ng g⁻¹ soil h⁻¹ at day 7, before gradually dropping to 0.05 ng g⁻¹ soil h⁻¹ at day 21 (FIG. 7a). This behaviour reflects the initially high N availability, which depletes over time. Compared to the uninhibited soil, treatment with DMP slowed down the N₂O formation depending on the application rate. Thus, with 0.5 mol % of DMP the N₂O production rate was with 0.32 ng g⁻¹ soil h⁻¹ at day 1 approximately 50% of that of the uninhibited soil. Using DMP at the higher application rates of 2.5 mol % and 5 mol %, the N₂O production rate was approximately 67% of that in the absence of the inhibitor. In contrast, soil treated with 4-MPT produced N₂O at a rate of only 0.06 ng g⁻¹ soil h⁻¹ at day 1 in the case of the lowest application rate, otherwise the amount of N₂O remained below the detection limit over the duration of the experiment, clearly revealing that 4-MPT is considerably more effective in reducing N₂O formation than DMP.

In the more acidic soil C the reduced amount of NH₃ available for oxidation by AMO due to protonation led to a reduced N₂O production rate of the uninhibited soil, compared to soil B (FIG. 7b). Soil treated with 0.5 mol % and 2.5 mol % of DMP did not show a significantly reduced N₂O formation rate in the first two weeks (P>0.05) compared to the uninhibited soil. Only at the highest application rate of DMP, a gradual reduction of the N₂O production rate to 0.095 ng g⁻¹ soil h⁻¹ at day 21 was found. In comparison, soil treated with 0.5 mol % of MPT produced about 0.12 ng h¹ of N₂O across the duration of the incubation, which is just 25% of the amount released from the uninhibited soil. At the higher 4-MPT application rates the production of N₂O was nearly completely suppressed.

The most acidic soil D produced the lowest amount of N₂O, ranging from 0.08 to 0.14 ng g⁻¹ soil h⁻¹ throughout the experiment (FIG. 7c). No significant difference of the N₂O production rates between the uninhibited soil and soil treated with DMP was found (P>0.05). In contrast, soil treated with 4-MPT at 0.5 mol % slowed down N₂O production by 60%, whereas 4-MPT at the two higher application rates reduced the N₂O production rate to practically zero from the start of the experiment, indicating that the inhibitory performance of 4-MPT is essentially pH-independent at these concentrations.

Soil E (pH=7.5) was collected from an agricultural recultivation site of a former opencast brown coal mine. Recultivation soils are known to maintain a low total N content over several decades. This lack of N retention capacity resulted in an unusual N₂O profile in the uninhibited soil, where the N₂O production rate rapidly increased within the first five days following fertiliser application, reaching a maximum of 2.33 ng g⁻¹ soil h⁻¹ at day 5, before declining again to practically zero at day 14, indicating a depleted N availability (FIG. 7d). Since N₂O formation correlates with the nitrification rate, this behaviour indicates a very high N nitrification/denitrification activity of this soil, which quickly adapts to N fertilization. Treatment with DMP and 4-MPT led to a dampening of the N₂O production, with 4-MPT being considerably more effective than DMP in the first five days. Beyond that timepoint, the rate of N₂O production in the presence of either inhibitor remained extremely low.

TABLE 9
Results of soil incubation studies in soils B-E to determine N₂O production by 4-MPT
and DMP at different concentrations of inhibitor.

| | Day 1 | Day 3 | Day 5 | Day 7 | Day 14 | Day 21 |
|---|---|---|---|---|---|---|
| | Soil B | | | | | |
| Untreated | 0.007 ± 0.007 | 0.008 ± 0.008 | 0.000 ± 0.000 | −0.021 ± 0.021 | 0.000 ± 0.000 | 0.000 ± 0.000 |
| Fertiliser | 0.681 ± 0.044 | 0.282 ± 0.023 | 0.204 ± 0.016 | 0.191 ± 0.009 | 0.108 ± 0.018 | 0.048 ± 0.006 |
| only | | | | | | |
| Fertiliser + | 0.318 ± 0.030 * | 0.189 ± 0.006 | 0.151 ± 0.015 | 0.086 ± 0.043 | 0.136 ± 0.006 | 0.019 ± 0.019 |
| DMP 0.5 | | | | | | |
| mol % | | | | | | |
| Fertiliser + | 0.224 ± 0.013 * | 0.124 ± 0.008 | 0.060 ± 0.030 | 0.045 ± 0.023 | 0.113 ± 0.006 | 0.127 ± 0.000* |
| DMP 2.5 | | | | | | |
| mol % | | | | | | |
| Fertiliser + | 0.209 ± 0.054 * | 0.067 ± 0.017 * | 0.058 ± 0.014 * | 0.020 ± 0.010 ** | 0.052 ± 0.003 | 0.037 ± 0.019 |
| DMP 5 | | | | | | |
| mol % | | | | | | |
| Fertiliser + | 0.058 ± 0.004 * | 0.009 ± 0.009 * | 0.000 ± 0.000 * | −0.013 ± 0.013 ** | 0.008 ± 0.008 | 0.011 ± 0.011 |
| 4-MPT 0.5 | | | | | | |
| mol % | | | | | | |
| Fertiliser + | 0.007 ± 0.007 * | 0.000 ± 0.000 * | −0.007 ± 0.007 * | −0.019 ± 0.019 ** | 0.000 ± 0.000^{#} | 0.000 ± 0.000 |
| 4-MPT 2.5 | | | | | | |
| mol % | | | | | | |
| Fertiliser + | 0.000 ± 0.000 * | 0.000 ± 0.000 * | 0.000 ± 0.000 * | −0.048 ± 0.026 ** | 0.000 ± 0.000^{#} | 0.000 ± 0.000 |
| 4-MPT 5 | | | | | | |
| mol % | | | | | | |
| | Soil C | | | | | |
| Untreated | 0.013 ± 0.013 | 0.000 ± 0.000 | 0.009 ± 0.009 | 0.027 ± 0.014 | 0.043 ± 0.003 | 0.052 ± 0.003 |
| Fertiliser | 0.283 ± 0.004 | 0.198 ± 0.003 | 0.168 ± 0.003 | 0.163 ± 0.002 | 0.243 ± 0.020 | 0.229 ± 0.008 |
| only | | | | | | |
| Fertiliser + | 0.283 ± 0.008 | 0.183 ± 0.003 | 0.081 ± 0.041 | 0.162 ± 0.037 | 0.105 ± 0.007 | 0.101 ± 0.011 ** |
| DMP 0.5 | | | | | | |
| mol % | | | | | | |
| Fertiliser + | 0.276 ± 0.006 | 0.161 ± 0.006 | 0.133 ± 0.006 | 0.113 ± 0.005 * | 0.086 ± 0.006 | 0.089 ± 0.005 ** |
| DMP 2.5 | | | | | | |
| mol % | | | | | | |
| Fertiliser + | 0.228 ± 0.017 | 0.125 ± 0.006 ** | 0.099 ± 0.008 * | 0.098 ± 0.010 | 0.085 ± 0.009 * | 0.095 ± 0.000 * |
| DMP 5 | | | | | | |
| mol % | | | | | | |
| Fertiliser + | 0.119 ± 0.011 ** | 0.062 ± 0.001 ***^{#} | 0.059 ± 0.007 ** | 0.054 ± 0.006 ** | 0.119 ± 0.010 | 0.107 ± 0.008 ** |
| 4-MPT 0.5 | | | | | | |
| mol % | | | | | | |
| Fertiliser + | 0.042 ± 0.04 ***^{##} | 0.000 ± 0.000 ***^{##} | 0.011 ± 0.011 *^{#} | 0.031 ± 0.003 *** | 0.012 ± 0.008 *^{#} | 0.019 ± 0.010 *** |
| 4-MPT 2.5 | | | | | | |
| mol % | | | | | | |
| Fertiliser + | 0.017 ± 0.017 *^{##} | 0.000 ± 0.000 ***^{##} | 0.000 ± 0.000 ***^{#} | 0.024 ± 0.013 * | 0.023 ± 0.007 *^{#} | 0.000 ± 0.000 **^{###} |
| 4-MPT 5 | | | | | | |
| mol % | | | | | | |
| | Soil D | | | | | |
| Untreated | 0.018 ± 0.018 | 0.083 ± 0.011 | 0.049 ± 0.005 | 0.080 ± 0.006 | 0.043 ± 0.006 | 0.067 ± 0.016 |
| Fertiliser | 0.128 ± 0.008 | 0.144 ± 0.013 | 0.081 ± 0.011 | 0.124 ± 0.010 | 0.076 ± 0.004 | 0.122 ± 0.010 |
| only ^{[a]} | | | | | | |
| Fertiliser + | 0.131 ± 0.001 | 0.133 ± 0.003 | 0.063 ± 0.003 | 0.112 ± 0.008 | 0.072 ± 0.009 | 0.098 ± 0.011 |
| DMP 0.5 | | | | | | |
| mol % | | | | | | |
| Fertiliser + | 0.073 ± 0.037 | 0.120 ± 0.005 | 0.067 ± 0.006 | 0.115 ± 0.012 | 0.070 ± 0.006 | 0.063 ± 0.003 |
| DMP 2.5 | | | | | | |
| mol % | | | | | | |
| Fertiliser + | 0.114 ± 0.015 | 0.112 ± 0.006 | 0.039 ± 0.021 | 0.102 ± 0.014 | 0.056 ± 0.004 | 0.069 ± 0.007 |
| DMP 5 | | | | | | |
| mol % | | | | | | |
| Fertiliser + | 0.029 ± 0.029 | 0.058 ± 0.009 * | 0.004 ± 0.004 | 0.037 ± 0.021 | 0.037 ± 0.005 *^{#} | 0.045 ± 0.014 |
| MPT 0.5 | | | | | | |
| mol % | | | | | | |
| Fertiliser + | 0.015 ± 0.010 * | 0.020 ± 0.010 *^{#} | 0.000 ± 0.000 | 0.037 ± 0.018 | 0.000 ± 0.000 *^{#} | 0.000 ± 0.000 * |
| MPT 2.5 | | | | | | |
| mol % | | | | | | |
| Fertiliser + | 0.000 ± 0.000 * | 0.027 ± 0.013 * | 0.000 ± 0.000 | 0.020 ± 0.011 * | 0.000 ± 0.000 *^{#} | 0.000 ± 0.000 *^{#} |
| MPT 5 | | | | | | |
| mol % | | | | | | |
| | Soil E | | | | | |
| Untreated | 0.026 ± 0.014 | 0.000 ± 0.000 | 0.000 ± 0.000 | 0.000 ± 0.000 | 0.000 ± 0.000 | 0.000 ± 0.000 |
| Fertiliser | 0.207 ± 0.018 | 0.680 ± 0.069 | 2.334 ± 0.972 | 0.059 ± 0.030 | 0.009 ± 0.009 | 0.000 ± 0.000 |
| only | | | | | | |
| Fertiliser + | 0.053 ± 0.002 * | 0.037 ± 0.006 * | 0.015 ± 0.008 | 0.000 ± 0.000 | 0.000 ± 0.000 | 0.000 ± 0.000 |
| DMP 0.5 | | | | | | |
| mol % | | | | | | |
| Fertiliser + | 0.080 ± 0.009 * | 0.014 ± 0.014 * | 0.000 ± 0.000 | 0.000 ± 0.000 | 0.008 ± 0.008 | 0.000 ± 0.000 |
| DMP 2.5 | | | | | | |
| mol % | | | | | | |
| Fertiliser + | 0.045 ± 0.011 * | 0.011 ± 0.011 * | 0.000 ± 0.000 | 0.000 ± 0.000 | 0.007 ± 0.007 | 0.000 ± 0.000 |
| DMP 5 | | | | | | |
| mol % | | | | | | |
| Fertiliser + | 0.007 ± 0.007 * | 0.011 ± 0.011 * | 0.000 ± 0.000 | 0.000 ± 0.000 | 0.000 ± 0.000 | 0.000 ± 0.000 |
| 4-MPT 0.5 | | | | | | |
| mol % | | | | | | |
| Fertiliser + | 0.008 ± 0.008 * | 0.008 ± 0.008 * | 0.000 ± 0.000 | 0.000 ± 0.000 | 0.000 ± 0.000 | 0.000 ± 0.000 |
| 4-MPT 2.5 | | | | | | |
| mol % | | | | | | |
| Fertiliser + | 0.012 ± 0.012 * | 0.000 ± 0.000 * | 0.000 ± 0.000 | 0.000 ± 0.000 | 0.000 ± 0.000 | 0.000 ± 0.000 |
| 4-MPT 5 | | | | | | |
| mol % | | | | | | |

^{[a]} The measurements were performed as described earlier, and production rates were calculated according to the equation provided in the experimental section above. All fertiliser treatments were at an application rate of (NH₄)₂SO₄ = 50 mg kg⁻¹ soil. Mean values (n = 3); errors are standard errors of the mean of biological replicates.
Statistical significance:
P < 0.05 (*), P < 0.01 (**), P < 0.001 (***) when comparing inhibitor treatments to the control treatment with (NH₄)₂SO₄ alone, P < 0.05 (^{#}), P < 0.01 (^{##}), P < 0.001 (^{###}), respectively, when comparing inhibitor treatments to the highest concentration of the DMP treatment. Statistical analyses were performed on raw N₂O production rates (μg g⁻¹ soil h⁻¹) with GraphPad Prism 9.5.0 (2-way ANOVA) multiple comparison Tuckey HSD test.

1. A compound of Formula (I) when used for inhibiting nitrification: 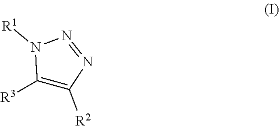
wherein:
R¹ is CH₂C≡CA, wherein A is H, methyl or ethyl;
R² is CₙH₂ₙ₊₁;
R³ is CₘH₂ₘ₊₁;
and wherein n and m+n are each independently 1, 2, 3, 4, 5 or 6 and m is 0, 1, 2, 3, 4 or 5.

2. The compound of claim 1, wherein A is H.

3. The compound of claim 1, wherein n is 1.

4. The compound of claim 1, wherein m is 0.

5. The compound when of claim 1, wherein the compound binds irreversibly to a nitrification enzyme.

6. The compound of claim 1, wherein the compound irreversibly deactivates a nitrification microorganism.

7. The compound of claim 1, wherein nitrification activity of N. europaea or N. multiformis increases from step (ii) to step (iv) by less than 10% on the following test:
i. ammonium sulphate and the compound are added to N. europaea or N. a multiformis cells in sodium phosphate buffer;
ii. NO₂⁻ production is measured via a Griess assay;
iii. the cells are then washed with buffer and ammonium sulphate added;
iv. NO₂⁻ production is again measured using a Griess assay.

8. A compound of Formula (I) when used for inhibiting nitrification: 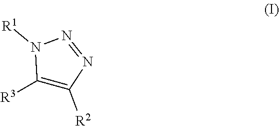
wherein:
R¹ is CH₂C≡CH;
R² is methyl; and
R³ is H.

9. A composition when used for inhibiting nitrification in soil, comprising a compound of Formula (I) as defined in claim 1 and at least one agriculturally acceptable adjuvant or diluent.

10. The composition when used of claim 9, further comprising a urease inhibitor.

11-20. (canceled)

21. A composition when used for inhibiting nitrification in soil, comprising a compound of Formula (I) as defined in claim 8, and at least one agriculturally acceptable adjuvant or diluent.

22. The composition when used of claim 21, further comprising a urease inhibitor.

23. A composition comprising a fertilizer, optionally a urea- or ammonium-based fertilizer, and a compound of Formula (I) as defined claim 1.

24. The composition of claim 23, further comprising a urease inhibitor.

25. A composition comprising a fertilizer, optionally a urea- or ammonium-based fertilizer, and a compound of Formula (I) as defined claim 8.

26. The composition of claim 25, further comprising a urease inhibitor.

27. The composition of claim 1, when used to inhibit the nitrification activity of a nitrification microorganism.

28. The composition of claim 1, when used to deactivate a nitrification enzyme.

29. The composition of claim 8, when used to inhibit the nitrification activity of a nitrification microorganism.

30. The composition of claim 8, when used to deactivate a nitrification enzyme.
